(19) United States
(12) Patent Application Publication
(10) Pub. No.: US 2007/0065486 A1
(43) Pub. Date: Mar. 22, 2007

(54) Migraine remedy

(75) Inventors: Sirichai Chayasirisobhon, Yorba Linda, CA (US); Larry E. Stenswick, Rangiora (NZ)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: MigCo Limited, Rangiora (NZ)

(21) Appl. No.: 10/557,932

(22) PCT Filed: May 21, 2004
(86) PCT No.: PCT/NZ04/00095

§ 371 Date: Sep. 28, 2006

(51) Int. Cl. A61K 36/185; A61K 31/7048; A61K 31/353; A61K 31/375; A61K 31/355

(52) U.S. Cl. 424/439; 514/27; 514/456; 424/725; 514/458; 514/474

(57) ABSTRACT

The invention relates to uses and methods of treating migraine with a plant extract that includes a therapeutically effective dose of a mixture of flavonoids rich in proanthocyanidis. Methods and uses are described to substantially prevent migraine from occurring; reduce the frequency of migraine or reduce the severity of migraine symptoms.

### TECHNICAL FIELD

The present invention relates to uses and methods of a plant extract that includes a therapeutically effective dose of a mixture of flavonoids rich in proanthocyanidins for preventing, reducing the frequency or reducing the severity of migraine. More specifically, the present invention relates to uses and methods of a plant extract that includes a therapeutically effective dose of a mixture of flavonoids rich in proanthocyanidins from the bark of the species Pinus for treatment of migraine (prevention, reduction in frequency or reduction in severity) in humans.

### BACKGROUND ART

The use of various compositions for treatment of migraine has been the subject of an increasing number of studies during the past two decades.

Migraine is a common, chronic, incapacitating neurovascular disorder, characterised by attacks of severe headache, autonomic nervous dysfunction and in some patients, an aura involving neurologic symptoms¹⁷. Migraine disorder is often familial³⁰ and defined as episodic attacks of headache lasting 4 to 72 hours with two or more of the following symptoms:
Unilateral pain
Throbbing
Aggravation on movement
Pain of moderate or severe intensity

And one of the following symptoms:
Nausea or vomiting
Photophobia or phonophobia¹⁷.

It is generally accepted that attacks are preceded by constriction of the cranial arteries usually with resulting prodromal sensory (especially ocular) symptoms and commence with the vasodilation that follows.

For persons suffering from regular migraine attack, the disruption on normal activities such as working can be severe. For most sufferers, work is either missed altogether due to migraine attack stopping all such activity or work performance is severely impinged. According to one reference²³, cost associated with lost work time due to migraine attack is estimated as being $US 6,864 per working male per year and $US 3,600 per working female per year. In another reference¹⁶, it is estimated that 18% of woman and 6% of men in the USA are affected by migraine.

The prior art has attempted to address the cause of migraine at length. A wide variety of causes have been suggested however it still appears that the exact mechanism is not certain¹⁶.

Migraine is episodic and varies among patients. This variability is likely to be due to the dysfunction of an ion channel in the aminergic brain stem nuclei that normally modulates sensory input and exerts neural influences on cranial vessels¹⁷.

A large number of treatments have been considered for acute migraine. A number have also been considered for prophylactic treatment of migraine¹⁷.

Useful references in relation to this area discuss the breadth of potential treatments to prevent migraine^{16,17,31}.

Migraine drug treatments are broadly split into two groups. Those for treatment of acute attacks of migraine and those for preventation of migraine³¹.

In summary, pharmaceutical drugs for treatment of acute attacks include triptans (serotonin receptor agonists), ergot alkaloids and derivatives, antiemetics, NSAIDs and nonopiate analgesics, combination analgesics, barbiturate hypnotics, opiate analgesics and others including corticosteroids, isometheptene compound and Lidocaine³¹.

Disappointingly from a headache sufferer's point of view, almost all of the above treatments involve mild to moderate side effects and/or mixed levels of efficacy^{16,17,31}. Some treatments cause a range of serious adverse reactions. One study found that over two-thirds of migraine sufferers that took part in a study had delayed or avoided taking a current prescription medication because of concerns about adverse effects³⁴.

The above factors mean that for light to moderate migraine sufferers, treatment by any of the above drugs is often avoided as the doses required to treat acute attacks of migraine cause marked and intolerable side effects¹⁷.

A further problem of the above pharmaceutical treatments is that such drugs are very expensive. By way of example, two drugs widely used for treatment of acute migraine, Imitrex™ (which accounts for 65% of the US market sales), costs approximately $US15 to $US17 per dose, while Zomig™ costs approximately $US17 to $US29.90 per dose³².

Preventative drug treatments for migraine include antiepileptics, antidepressants, beta-blockers, calcium channel blockers, NSAIDs, serotonin antagonists³¹. Other therapies include use of feverfew, magnesium and vitamin B2³¹. The goals of preventative therapy (or prophylactic therapy) are to reduce the frequency, severity and duration of migraine symptoms; to improve function and to reduce disability.

Like acute treatments, the side effects and efficacy of preventative treatments is also a concern³¹.

Some migraine sufferers try to avoid the trigger that can set off an acute migraine attack. These vary be individual and over time and can include dozens of triggers such a stress or tension, missing meals, fatigue, lack of sleep, smoke or some sort of odour.

Alternative or complementary or natural methods for preventative treatment of migraine have been considered although often the results lack scientific proof or accuracy. Options include: aerobic exercise; isometric neck exercises; biofeedback; acupuncture; as well as natural remedies including feverfew, riboflavin and magnesium^{7,16,18-21}.

Feverfew (Tanacetum parthenium) in particular has been given significant attention for use as a prophylactic migraine treatment. Feverfew is a plant that is claimed to have main active ingredients of sesquiterpene lactones, particularly parthenolide, which inhibits serotonin release by human platelets in vitro^{8,9}. However, use of this composition does have potential complications. As feverfew has been claimed to relax uterine smooth muscle, it is to be avoided during pregnancy. Discontinuation can result in a post-feverfew syndrome including nervousness, insomnia, joint stiffness and pain. Other side effects also include mouth ulceration and a more widespread oral inflammation associated with loss of taste¹⁶.

Studies on the effectiveness of feverfew have also had mixed results. In some studies, results have shown that feverfew gives useful results^{10,16}, however in other studies^{11,12,13,16}, the results are less conclusive and in fact one case failed to show any effect.

A more recent Cochrane review of five trials on Feverfew efficacy (343 patients) concluded that overall results were mixed and there was insufficient evidence from randomised, double blind trials to suggest an effect of feverfew over and above placebo for preventing migraine³⁵.

Riboflavin (vitamin B2)—chemically known as 7,8-dimethyl-10 (1′-D-ribityl) isoalloxazine⁸—has been shown to have some low level efficacy¹⁶. In one study, in comparison to placebo treatments, the attack frequency, headache days and migraine index all showed a statistically significant improvement for subjects taking riboflavin. Of note however was the fact that the dose used in the trial was massive in comparison to RDA limits i.e. 400 mg per dose in the trial compared to 1.3 mg per dose per day for RDA requirements¹⁸. It is noted that only about 25 mg of riboflavin can be absorbed in a single oral dose²¹. The implications of this significantly higher dose are not considered in the study¹⁶ however given the discrepancy with RDA guidelines, it is possible that adverse effects may occur. Side effects for riboflavin taking subjects in the study¹⁶ included diarrhoea and polyuria. A further publication²⁰ shows mixed results for subjects taking riboflavin from positive results to no effect at all. One subject even complained that taking riboflavin caused bad headaches.

A further remedy utilises magnesium, a common mineral in diets¹⁹. Low levels have been found in some patients with migraines. Taking magnesium does have complications with kidney or heart diseases and also, as magnesium competes with calcium for absorption, magnesium supplements can cause a calcium deficiency. Further side effects from magnesium supplementation includes diarrhoea.

Combination therapies of the above compounds are also available including ‘Migrahealth® triple therapy’ which is a supplement that contains two high dose forms of magnesium, a high dose of riboflavin and feverfew extract²⁹.

Yet a further alternative remedy for migraines relates to the use of Pelasites hybridus or Butterbur. This is a herb that has been used medicinally for centuries to treat plague, fever, cough, asthma and skin wounds. Some evidence exists that by taking an extract of Butterbur, patients experience a reduction in migraine frequency. Side effects are noted however. Butterbur contains high levels of cancer causing compounds and even though one company says it has developed a method to remove these compounds, some doctors who specialise in the treatment of headache and severe pain question whether the safety of even a modified form of the drug has been established. Active constituents of this drug have been identified as two sesquiterpenes, petasin and isopetasin. Flavonoids are thought to be present in Butterbur extracts but the content of proanthocyanidins is not in any rich quantities or attributed to the activity noted^{27,28}.

In the inventor's experience, flavonoids and/or antioxidants are not known to have effects on migraine. Some compositions rich in phenolic flavonoids such as red wine have in fact been considered as activators of migraine attacks^{13,14,15,33}.

One patent application, WO 99/23881 teaches of the use of dietary supplementation of serotonin and norepinephrine precursors in the treatment of migraine headache. Serotonin and norepinephrine precursors are described as the active components in this supplement. The specification further describes the optional use of bioflavonoids as ancillary compounds with the serotonin and norepinephrine precursors. The ancillary effect of the bioflavonoids is described as strengthening outer cell membranes and helping to stabilise cell surfaces—not to treat the symptoms in itself. There is no teaching or even suggestion of bioflavonoid anti-migraine activity and several example formulations in the specification do not contain any bioflavonoids.

Patent application WO 99/17612 describes a serotonin/antioxidant mixture for pain treatment including migraine. The invention described refers to co-administration to achieve the protection of serotonin in the gastrointestinal tract.

Serotonin is specified as the active ingredient to treat migraine. No description or suggestion is included for the use of antioxidants alone in migraine treatment.

Patent application WO 99/61028 (the '028 publication) describes use of specific isoflavonoid compounds including genistein, daidzein, biochanin A, forononetin, O-desmethylangolensin, glycitein, equol and dihydrodaidzein and their conjugates for the treatment of migraine. The treatment effectiveness is ascribed to the estrogenic effects that are a particular property of the iso-flavonoids. No teaching is made of use of flavonoids that do not have estrogenic properties but that are strong antioxidants. The '028 publication does not provide any data for the claims such as human trials showing efficacy. Given that the exact mechanism of action for migraines is uncertain, it is arguable that it would not be obvious to a person skilled in the art that a composition including iso-flavonoids would have the claimed efficacy.

Patent application WO 03/074065 describes use of a composition containing a particular type of flavonoids compound that it refers to as free-B-ring flavonoids to treat COX-2 mediated diseases and conditions, which the specification describes as including migraine. Free-B-ring flavonoids are rare, as identified within the specification and generally not found from water based extraction methods. Further, the specification does not describe that this assertion of treatment of migraine actually occurs as there is no information to show efficacy such as experimental trials. Nor would it be obvious that efficacy would be seen as the mechanisms for how migraine occur is unknown. Hence it would not necessarily follow that a COX-2 mediator would treat migraine.

EP 906761 teaches of the use of a composition prepared by extracting phytochemicals from plant matter for treatment of various cancers, pre- and post menstrual syndromes and various other disorders. A generalised statement is included that, as isoflavones, lignans and saponins are known individually to effect neurological and immunological symptoms, it is the patentee's belief that the composition described in EP 906761 has a neurological and immunological effect. No further basis is described for this belief or claim in relation to migraine treatment. As described above, the mechanism behind the cause of migraine is not well established and hence it would not be obvious that the composition described in EP 906761 would in fact have an effect against migraine.

U.S Pat. No. 6,517,832 (the '832 patent) describes a prophylactic treatment for migraine consisting of daily administration of two formulations, one including peptides and probiotics and a second formulation including a major amount of active components including malic acid, sylibum marianum, acetyl-L-cysteine, copper chelate, zinc gluconate, asparatate and bromelain. A minor amount of plant derivatives is also included in the second formulation which includes derivatives from beet root, water cress, celery, dandelion, capsicum and artichoke extract. The '832 patent describes the minor amount of plant derivatives as useful adjuncts for optimal effectiveness, but not essential to an effective unit dosage—rather the ‘major’ amounts are key to the therapy.

Chemical abstract number 131:63300 teaches of use of the leaves from Caragana chamiagu for medicinal purposes. The abstract notes that roots of Caragana chamlagu have been used as a migraine remedy in folk medicines of Korea. Some flavonoid compounds were identified in the mixture. No results are described however showing a medicinal effect on migraines such as prevention of migraine symptoms.

The website:

http://www.susanlovemd.com/takecharge/alternatives/herbs/grapeseedextract.htm describes various traditional uses for grape seed preparations to stimulate liver function and metabolism; and to treat migraine, gout, and arthritic pain. The majority of the discussion teaches of treatments of breast cancer. Migraine is only mentioned in passing as a traditional use with no supporting information showing a medicinal effect on migraine such as prevention of migraine.

The reference Burke B. E. et al ‘Randomized, controlled trial of phytoestrogen in the treatment of menstrual migraine²⁶’ describes a study which tested the use of phytoestrogen compounds as a prophylactic treatment of menstrual migraine. The aim of the phytoestrogen treatment was to prevent estrogen and progestin fluctuations and thus avoid menstrual migraine symptoms. This treatment is thus quite specific to a form of migraine and only female patients.

Serious chronic headaches are also an illness in themselves for some patients. Chronic daily headache (CDH) affects approximately 4 to 5% of the population and encompasses a number of different diagnoses including transformed migraine, chronic tension-type headache (TTH), new-onset daily persistent headache, and hemicrania continua²². Like migraine treatments, the goals of prophylactic therapy are to reduce the frequency, severity and duration of headache attacks; to improve responsiveness to treatment of acute attacks; to improve function; and to reduce disability. Similar pharmaceutical drugs in CDH treatments have been employed to migraine headache treatments²².

It is therefore an object of the present invention to provide an improved migraine and/or CDH treatment.

It is a further object of the present invention to address the foregoing problems or at least to provide the public with a useful choice.

All references, including any patents or patent applications cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents form part of the common general knowledge in the art, in New Zealand or in any other country.

It is acknowledged that the term ‘comprise’ may, under varying jurisdictions, be attributed with either an exclusive or an inclusive meaning. For the purpose of this specification, and unless otherwise noted, the term ‘comprise’ shall have an inclusive meaning—i.e. that it will be taken to mean an inclusion of not only the listed components it directly references, but also other non-specified components or elements. This rationale will also be used when the term ‘comprised’ or ‘comprising’ is used in relation to one or more steps in a method or process.

Further aspects and advantages of the present invention will become apparent from the ensuing description which is given by way of example only.

### DISCLOSURE OF INVENTION

The present invention broadly relates to methods of treating migraine and/or CDH by use of a novel composition for such treatments.

For the purposes of this description, the term migraine is used with reference to symptoms exhibited from the malady termed ‘migraine’ as well as serious chronic headache conditions such as chronic daily headache (CDH) and related disorders.

The term mixture of ‘flavonoids rich in protein’ refers to compositions containing large numbers of flavonoid compounds, predominantly from the group of proanthocyanidins (also termed procyanidins) being the active ingredients in the composition.

For the purposes of this specification, the term ‘human’ will be referred to interchangeably with the terms, ‘patient’, ‘subject’, ‘sufferer’ and the like. This should not be seen as limiting.

According to a further aspect of the invention, there is provided the use of a plant extract that includes a therapeutically effective dose of a mixture of flavonoids rich in proanthocyanidins in the manufacture of a medicament to prevent migraine in a human in need thereof.

According to a further aspect of the invention, there is provided the use of a plant extract that includes a therapeutically effective dose of a mixture of flavonoids rich in proanthocyanidins in the manufacture of a medicament to reduce the frequency of migraine symptoms in a human in need thereof.

According to one aspect of the present invention, there is provided the use of a plant extract that includes a therapeutically effective dose of a mixture of flavonoids rich in proanthocyanidins in the manufacture of a medicament to reduce the severity of migraine symptoms in a human in need thereof.

According to a further aspect of the present invention, there is provided a method of treatment of a human in need thereof by administration of a plant extract that includes a therapeutically effective dose of a mixture of flavonoids rich in proanthocyanidins to prevent migraine.

According to a further aspect of the present invention, there is provided a method of treatment of a human in need thereof by administration of a plant extract that includes a therapeutically effective dose of a mixture of flavonoids rich in proanthocyanidins to reduce the frequency of migraine.

According to a further aspect of the present invention, there is provided a method of treatment of a human in need thereof by administration of plant extract that includes a therapeutically effective dose of a mixture of flavonoids rich in proanthocyanidins to reduce the severity of migraine symptoms.

According to a further aspect of the present invention there is provided a plant extract that includes a therapeutically effective dose of a mixture of flavonoids rich in proanthocyanidins that prevents migraine.

According to a further aspect of the present invention there is provided a plant extract that includes a therapeutically effective dose of a mixture of flavonoids rich in proanthocyanidins that reduces the frequency migraine symptoms.

According to a further aspect of the present invention there is provided a plant extract that includes a therapeutically effective dose of a mixture of flavonoids rich in proanthocyanidins that reduces the severity of migraine symptoms.

It has been found unexpectedly by the applicant that by administration of a plant extract that includes a therapeutically effective dose of a mixture of flavonoids rich in proanthocyanidins as the main active ingredient, a subject suffering from regular acute migraine observes a significant decline in the severity of symptoms exhibited; a reduction in the frequency with which migraine are exhibited and even substantial prevention of migraine.

Preferably the flavonoid source is a plant although this should not be seen as limiting as it should be appreciated by those skilled in the art that the compounds of the present invention may also be synthesised using non-natural processes. More preferably the extract plant source is bark. Yet more preferably the bark is a pine bark. It will be appreciated that other flavonoid sources can be used besides pine bark, for example grape seed and green tea. For the purposes of this specification, further reference will be made in terms of pine bark. This should not be seen as limiting.

Preferably the symptoms affected are migraine headaches.

Preferably the subject suffers from regular severe and acute migraine.

It is the inventors understanding that the mode of action against migraine is likely to be multifactorial. Previous studies have shown that the radical scavenging capacity of flavonoids and particularly proanthocyanidins is several times higher than that of the vitamin antioxidants for a wide range of radicals³⁶. Flavonoids are a chemically diverse range of compounds containing different ring structures with different side chains and polymerisation states. This diversity greatly contributes to their radical scavenging ability and effects on enzyme systems in the body^{37,38}. Numerous studies in cell culture systems, animal models and humans have documented the diverse effects of flavonoids. These effects include vasodilatory, anti-hypertensive, and anti-inflammatory activities, inhibition of lipid and LDL oxidation; inhibition of platelet aggregation, and capillary permeability and fragility; effects on enzyme systems including phopholipase A2, cyclooxygenase, and lipoxygenase; influencing cell signalling transduction; metal chelation; glutathione metabolism; intracellular calcium levels; protection of vitamin C and E from oxidation; and cardio protective effects after ischemic reperfusion injury³⁹⁻⁴⁶.

A further complication in migraine treatment is that sufferers displaying migraine have different mechanisms involved that both trigger a migraine attack and determine the severity of such attacks. Hence it should be appreciated by a person skilled in the art that the degree of response by treatment will vary depending on the subject characteristics.

It is understood by the applicant that dose rates may vary depending on the metabolism level, age, weight, gender, or genetics of the subject and other biochemical factors, such as seasonal dietary requirements.

It will also be appreciated by those skilled in the art that a dosage lesser or greater than that above is also possible without harm. Flavonoid extracts rich in proanthocyanidins are generally non-toxic and have naturally occurring sources. Higher doses would not produce any toxic reactions to the subject and may in fact be advantageous for some subjects that require additional nutritional antioxidant treatment for reasons described above.

Pine bark extract in particular has been shown to be non-toxic; does not result in changes in any biochemical or haemoglobin index; and has a broad spectrum of actions against a wide variety of free radical events²⁵. A placebo trial (unpublished) has also confirmed non-toxicity as well as increased antioxidant mechanisms in the body.

By way of example, the dose is from 1 to 10 capsules per day, each capsule containing pine bark extract used in the above composition includes primarily flavonoid compounds rich in levels of proanthocyanidins and associated compounds found naturally in these extracts.

Preferably the pine bark extract composition as described above is an extract which exhibits antioxidant behaviour in vivo and in vitro.

Preferably, the pine bark extract used in the above composition is sourced from the bark of Pinus radiata (the Monterey pine or radiata pine).

Most preferably, the pine bark extract (from Pinus radiata bark), is extracted using a water-based process. One example process is that of NZ329658/U.S. Pat. No. 5,968,517 (the '517 patent), incorporated herein by reference. The extract of the '517 patent is a complex mixture of mainly flavonoids with some non-flavonoid compounds. It is the inventors understanding that the extract predominantly consists of proanthocyanidins including dimers, trimers, oligomers and polymers of catechin and epicatechin. Other phenolic compounds of Pinus radiata bark include: catechin; epicatechin; gallocatechin; quercetin; dihydroquercetin; myricetin; astringenin; pinosylvin; taxifolin; stilbenes; hydroxylstilbenes; phenolic acids; and combinations thereof.

Optionally, the composition, substantially as described above, further includes other antioxidant active components. These include vitamin C, vitamin E, and other known therapeutically active antioxidant compounds. Preferably, the vitamin E used is a natural vitamin E. The use of vitamin E and C is to administer to the patient further antioxidants that are readily absorbed by the patient.

In preferred embodiments, the composition as described above is delivered to a subject as a regular daily dosage. Preferably the dose is 1 to 10 capsules per day, each capsule containing approximately 120 mg pine bark extract, approximately 30 IU of natural vitamin E, and approximately 60 mg of vitamin C.

In a further option the composition, substantially as described above, is also formulated using components selected from the group including; fillers; excipients; modifiers; humectants; stabilisers; emulsifiers; and other known formulation components.

Preferably, the composition, substantially as described above, is administered in a form selected from the group including: a tablet; a capsule; a suppository; an injection; a suspension; a drink or tonic; a syrup; a powder; an ingredient in solid or liquid foods; a nasal spray; a sublingual wafer; a transdermal patch; a transdermal injection; and combinations thereof.

A major advantage found by subjects taking the composition of the present invention is that the subject is able to attend work/school more regularly i.e. participate in normal life activities. In some cases, subjects are able to attend work/school without missing a day compared to prior to trial of the above composition where many days were missed resulting in decreased productivity.

Trials suggest that headache frequency decreases by as much as 56% and headache severity decreases by as much as 33.3%. It should be appreciated by those skilled in the art that through further development, it is likely that these results will show even stronger reductions in frequency and severity as the dose and mechanisms become further established.

A further advantage of the present invention is that side effects appear to be minimal if present at all. This is a major improvement on the prior art where side effects are a common occurrence where the side effect can prevent treatment in mild to moderate cases, and can cause severe adverse reactions¹⁷.

It can be seen from the above description that by administration of a composition containing pine bark extract to a subject, the frequency of migraine is reduced, the severity of such symptoms are reduced, and migraine even substantially prevented.

### BRIEF DESCRIPTION OF DRAWINGS

Further aspects of the present invention will become apparent from the ensuing description which is given by way of example only and with reference to the accompanying drawings in which:

FIG. 1 Shows a graph comparing the mean MIDAS score assessed before and after the three month trial period;

FIG. 2 shows a graph comparing the mean number of headache days before and after the three month trial period;

FIG. 3 shows a graph comparing the mean symptom score before and after the three month trial period; and,

FIG. 4 shows a graph comparing the relationship between the percentage reduction and patient reported disability score at baseline.

### BEST MODES FOR CARRYING OUT THE INVENTION

The present invention will now be further described with reference to a study on the effects of the composition on migraine patients.

Patients and Methods

This was an uncontrolled, open-label study of 3 months' duration. For inclusion, patients had to have a long-term history of regular migraine attacks diagnosed according to International Headache Society criteria. Patients' migraine also had to be refractory to treatment with beta-blockers, antidepressants, anticonvulsants, or 5-HT receptor agonists. In order to reliably self-assess the impact of migraine in terms of number of days of lost and limited activity, patients were selected with high intellectual capacity. Written informed consent was obtained from each patient.

Ten female and two male patients aged 22 to 58 years (mean±standard deviation age, 41.1±13.2 years) fitting the suitability criteria were enrolled in the study. Patients exhibited a broad range of clinical presentations: headaches were variously described as left or right frontal, bilateral frontal, bilateral frontal/temporal, bilateral frontal/top, left temporal, left side, right parietal, or diffuse; age of onset varied from 6 to 45 years (mean 19.5±12.3 years); frequency varied from 2 to 30 per month (mean 9.8±7.8 per month); and duration varied from 1 to 3 days, although one patient reported headaches lasting as long as 7 days. Eight patients were receiving monotherapy involving one of gabapentin, exedrine, midrin, amitriptyline, or sumatriptan. Four patients were receiving combination therapy of gabapentin and clonazepam, nortriptyline and valproate, valproate and amitriptyline, or valproate and amitriptyline and rizatriptan. No changes in patients' medications were made during the study and patients were instructed to keep taking their medications.

The Migraine Disability Assessment Score (MIDAS) method was used to assess the treatment. The MIDAS assessment is a 5-item questionnaire designed to assess the headache-related disability for use in routine clinical practice. Scores are divided into grades I through IV with I indicating little disability over the past 3 months associated with low treatment need. Grade II indicates mild disability and moderate treatment need. Grades III (scores 11-20) and IV (scores>20) identify moderate to severe disability and high treatment need²⁴.

MIDAS scores of trial participants were assessed at baseline using a standard questionnaire⁷. This comprised of 5 scoring questions to assess the number of days of lost or limited productivity in the previous 3 months involving work, school, household work, and family, social and leisure activities. Two non-scoring questions were included to provide additional information relating to the number of headache days and headache severity over the previous 3 months.

Patients received 10 capsules per day of a commercial formulation of standardised pine bark extract (Enzogenol®), with each capsule containing approximately 120 mg of pine bark extract, approximately 30 IU of natural vitamin E and approximately 60 mg of vitamin C. Patients were supplied with 3 bottles (120 capsules/bottle) each month for 3 months and were instructed to take the capsules in the morning. A second MIDAS assessment was performed after 3 months and liver function tests were performed at baseline and after 3 months.

Statistical Analysis

Changes in MIDAS score, number of headache days and headache severity from baseline to the end of the treatment period were analysed using the Wilcoxon method. Spearman correlation was used to assess whether responsiveness to pine bark extract was correlated with migraine disability at baseline.

Results

Of the 12 patients who were enrolled in the study, 11 successfully completed the 3-month treatment period and were included for analysis. One patient discontinued treatment on day 11 after reporting no change in headache frequency and was not considered in the analysis. Tests at baseline and after 3 months confirmed that pine bark extract treatment did not affect liver function.

At baseline 8 of 11 patients had grade IV migraine and three had grade III migraine on the MIDAS scale. Following 3 months of therapy with pine bark extract 1200 mg/day, 3 patients remained at grade IV, two grade IV patients were re-graded to grade III and six grade IV and grade III patients were re-graded to grade I. Of the 3 patients who continued to demonstrate grade IV migraine, one showed a reduction in MIDAS score and in headache severity. The other 2 grade IV patients showed no improvement in MIDAS score, number of headache days, or headache severity and were classed as non-responders. The 8 patients who demonstrated a reduction in MIDAS score and were re-graded to a lower grade also showed reductions in both number of headache days and headache severity over the previous 3 months.

For the scoring component of the MIDAS assessment, total days of lost or limited activity due to migraine over a range of common activities were counted, with results summarised in Table 1. FIG. 1 shows that the mean MIDAS score for all patients was significantly reduced from 31.8 days to 15.7 days (p<0.005) whilst the mean MIDAS score when restricted to responders decreased significantly from 28.7 days to 9.2 days (p<0.005) following 3 months of therapy with pine bark extract 1200 mg/day. This was equivalent to a mean improvement of 50.6% and 67.9% respectively in patients' MIDAS scores.

TABLE 1
Effect of 3 months' therapy with pine bark extract 1200
mg/day on Migraine Disability Assessment (MIDAS) score.

| | | | | | Reduction | |
|---|---|---|---|---|---|---|
| | | | | | from | |
| | | | Baseline | 3 Months | baseline | |
| Days of work or school missed | | | 42 | 21 | 21 | (50.0%) |
| Days where productivity half or less | | | 59 | 28 | 31 | (52.5%) |
| Days household work not done | | | 104 | 51 | 53 | (51.0%) |
| Days household productivity half or less | | | 103 | 49 | 54 | (52.4%) |
| Days where social activities missed | | | 42 | 24 | 18 | (42.9%) |
| Total days (all patients, n II) | | | 350 | 173 | 177 | (50.6%) |
| MIDAS Score (days) | All Patients | Mean | 31.8 | 15.7 | 16.1* | (50.6%) |
| | (n = 11) | 95% CI | 18.9-44.8 | 1.0-30.4 | 8.2-23.9 | |
| | Responders | Mean | 28.7 | 9.2 | 19.5* | (67.9%) |

| | | | | |
|---|---|---|---|---|
| Only (n-9) | 95% CI | 14.3-43.0 | 0-22.4 | 11.8-27.1 |

*Indicates a significant difference (p < 0.005) between baseline and 3 months.

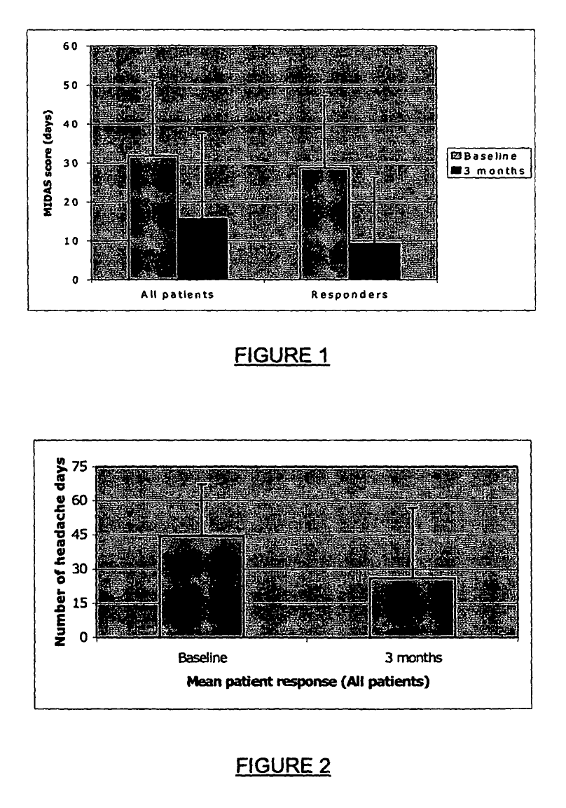

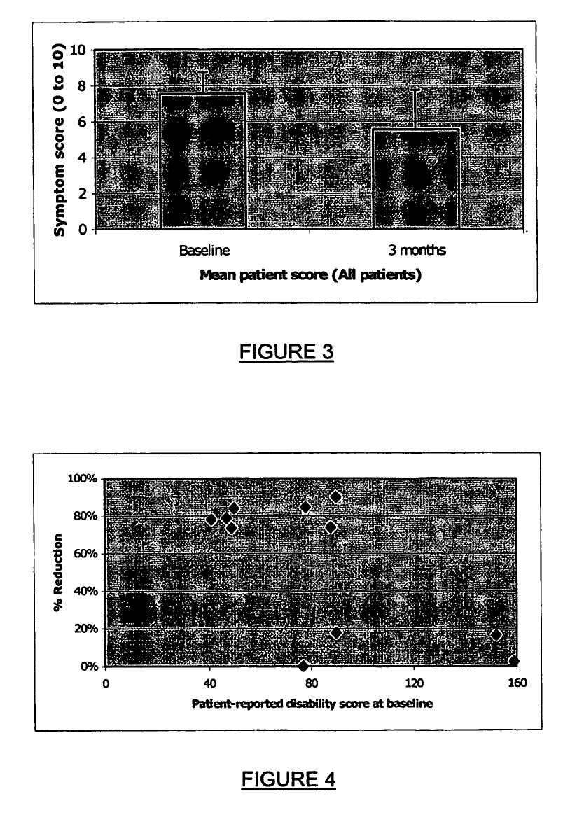

At baseline the mean number of headache days reported for the previous 3 months by all patients was 44.4 days whilst headache severity over the same period received a mean score of 7.5 (Table 2). FIGS. 2 and 3 show that following 3 months of therapy with pine bark extract 1200 mg/day the mean number of headache days reported by patients decreased significantly to 26.0 days (p<0.005) whilst headache severity also significantly decreased to a mean score of 5.5 (p<0.005), equivalent to reductions of 41.4% and 26.7% respectively.

When data was restricted to responders for analysis, the mean number of headache days and mean headache severity was significantly reduced from baseline by 56.0% and 33.3% respectively (p<0.005; Table 2).

TABLE 2
Effect of 3 months' therapy with pine bark extract 1200
mg/day on number of headache days and headache severity.

| | | | | | Reduction |
|---|---|---|---|---|---|
| | | | | | from |
| | | | Baseline | 3 Months | baseline |
| Number of headache | All Patients | Mean | 44.4 | 26.0 | 18.4* |
| days in previous 3 | (n = 11) | 95% CI | 28.9-59.8 | 5.3-46.7 | (41.4%) |
| months (days) | | | | | 7.7-29.0 |
| | Responders | Mean | 40.2 | 17.7 | 22.5* |
| | only (n = 9) | 95% CI | 25.4-55.0 | 0-36.4 | (56.0%) |
| | | | | | 11.5-33.6 |
| Severity of headaches | All Patients | Mean | 7.5 | 5.5 | 2.0* |
| over previous 3 months | (n = 11) | 95% CI | 6.7-8.4 | 4.1-7.0 | (26.7%) |
| (symptom score, 0 to 10) | | | | | 0.9-3.1 |
| | Responders | Mean | 7.2 | 4.8 | 2.4* |
| | only (n = 9) | 95% CI | 6.5-8.0 | 3.6-5.9 | (33.3%) |
| | | | | | 1.4-3.5 |

*Indicates a significant difference (p < 0.005) between baseline and 3 months.

In order to test the possibility that responsiveness to pine bark extract therapy was correlated to migraine disability at baseline, the number of headache days, headache severity score and MIDAS score were added for each patient to give a total patient-reported disability score at baseline and after 3 months of pine bark extract therapy. Spearman correlation analysis showed no significant correlation between percentage reduction in patient-reported disability score after 3 months and patient-reported disability score at baseline (FIG. 4).

Discussion

The therapy of migraine disorder is complicated by the variable responses seen among patients and by a lack of any cohesive experimental framework that synthesises our current understanding of migraine pathophysiology¹⁻⁵.

In terms of patient response, factors such as presence or absence of aura, duration of headache, severity and intensity of headache, and as yet undefined genetic and environmental factors all appear to impact on the efficacy of antimigraine drugs.

At the pathophysiological level there are several models that attempt to explain migraine. A vascular theory postulates that vasoconstriction occurs in the cranial blood vessels and pain results from vessel dilatation. A neuronal theory suggests that a “spreading depression” in the primary migraine reflects a central neuronal disorder. A neurovascular hypothesis suggests headache is the result of nociception from cephalic arteries and pericranial myofascial tissues. A brainstem theory proposes that the causative mechanism is located in the dorsal raphe nucleus of the brainstem; pain is triggered by secondary activation of nociceptive fibres of the trigeminal nerve that produce vasodilatation and inflammation. Yet another theory postulates that trigeminal nerve fibres become sensitised to stimuli in response to changes in norepinephrine from the locus ceruleus and neurotransmission of serotonin from the dorsal raphe nuclei.

In contrast to drugs either developed or used as therapy (as acute treatment therapy or prevention) for migraine such as subtype-selective 5-HT receptor agonists, or antidepressant and anticonvulsant drugs that have found some success in treating migraine, there is currently no experimental basis for the use of flavonoids as treatment of migraine. However, diverse biochemical and pharmacologic effects of flavonoids have been described and continue to be the subject of intense scientific scrutiny⁶. In vitro effects of flavonoids on smooth muscle contractility, nerve cell function and inflammation may be relevant in the present context of migraine, although further research is needed.

The finding in the present study that chronic migraine sufferers treated for 3 months with pine bark extract showed significant improvement in MIDAS score, headache frequency and headache severity suggests that pine bark extract may mitigate some as yet unknown mechanisms involved in a migraine attack. This is a potentially important finding as all patients had failed other pharmacologic therapies and therefore represent a challenging group.

Of 11 patients who completed the study, it is particularly noteworthy that 9 were responsive to pine bark extract, seven of these demonstrating a strong response. Although the 2 non-responders and a further 2 poor responders had higher combined patient-reported disability scores at baseline, analysis of percentage reduction in patient-reported disability scores after 3 months of pine bark extract therapy versus baseline patient-reported disability scores indicated there was no significant correlation.

These results show a substantial effect by pine bark extract on migraine frequency and severity due to the number of patients responding and the strong degree of response in the majority of responders.

Thus the above results show that the present invention provides a method of treating migraine that prevents, reduces the frequency, or reduces the severity of migraine symptoms. It should be appreciated by those skilled in the art that, as the composition is non-toxic and has few side effects, the treatment method provided is an improvement on many existing treatments where side effects are noted.

Aspects of the present invention have been described by way of example only and it should be appreciated that modifications and additions may be made thereto without departing from the scope thereof as defined in the appended claims.

### REFERENCES

1. Thomsen L L, Olesen J, Nitric oxide in primary headaches, Curr Opin Neurol 2001 June; 14(3):315-21.
2. Olesen J, Jansen-Olesen I, Nitric oxide mechanisms in migraine, Pathol Biol (Paris) 2000 September; 48(7 :648-57
3. Lee T J, Nitric oxide and the cerebral vascular function, J Biomed Sci 2000 January-February; 7(1):16-26.
4. Lassen L H, Ashina M, Christiansen I, Ulrich V, Grover R, Donaldson J, Olesen J, Nitric oxide synthase inhibition: a new principle in the treatment of migraine attacks, Cephalalgia 1998 January; 18(1):27-32.
5. Lassen L H, Thomsen L L, Olesen J, Histamine induces migraine via the H1-receptor. Support for the NO hypothesis of migraine, Neuroreport 1995 Jul. 31; 6(11):1475-9.
6. Houston D S, Vanhoutte P M, Serotonin and the vascular system. Role in health and disease, and implications for therapy. Drugs 1986 February; 31(2):149-63.
7. Mauskop A, Alternative therapies in headaches. Is there a role? Med Clin North Am 2001 July; 85(4):1077-84.
8. The Foundation to Health, 7 Essentials, http://www.7essentials.com/herbs/feverfew.shtml
9. Ernst E, Pittler M H, The efficacy and safety of feverfew (Tanacetum parthenium L.): an update of a systematic review, Public Health Nutr 2000 December; 3(4A):509-14.
10. Pfaffenrath V, Diener H C, Fischer M, Friede M, Henneicke-von Zepelin H H; Investigators, The efficacy and safety of Tanacetum parthenium (feverfew) in migraine prophylaxis—a double-blind, multicentre, randomised placebo-controlled dose-response study, Cephalalgia 2002 September: 22(7):523-32.
11. Pittler M H, Vogler B K, Ernst E, Feverfew for preventing migraine, Cochrane Database Syst Rev 2000; (3):CD002286.
12. Prusinski A, Durko A, Niczyporuk-Turek A, Feverfew as a prophylactic treatment of migraine, Neurol Neurochir Pol 1999; 33 Supp. 5:89-95.
13. Pattichis K, Louca L L, Jarman J, Sandler M, Glover V, 5-Hydroxytryptamine release from platelets by different red wines: Implications for migraine, Eur. J. Pharmacol. (Environ. Toxicol. Pharmacol. Sect.) 1995; vol. 292, no. 2, pp. 173-177.
14. Kwa niewska M, Kostka T, Drygas W, Red wine in medicine: panacea, fashion or . . . risk factor?, Przegl Lek 2000, vol. 57, pp 300-4.
15. Leira R, Rodriguez R, Diet and migraine, Rev Neurol 1996 May, vol. 24, no. 129, pp. 534-8.
16. Silberstein, S. D, Migraine: Preventative Treatment, Curr Med Res Opin Vol. 17 Suppl 1 2001 pp. 87-93.
17. Goadsby P. J. et al. ‘Migraine—Current Understanding and Treatment’, N Engl J Med, Vol. 346, No. 4, pp 257-270, 2002.
18. McCormick, D. B. Riboflavin, Amer Statement of Case for Nutritional Sciences, 2003.
19. Wong, C. Natural Migraine Relief, www.about.com, 2003.
20. Anon, Vitamin B-2 (Riboflavin), www.remedyfind.com, 2003.
21. Hogdon, J. Riboflavin, The Linus Pauling Institute, Micronutrient Information Center, Oregon State University, 2002.
22. Lake, A. E., Saper J. R. Chronic Headache—New Advances in Treatment Strategies, Neurology 2002; 59:S8-S13, American Academy of Neurology, 2002.
23. GlaxoSmithKline press release, Imitrex® Now Available in Tablet Form in U.S., www.pslgroup.com/dg950907.htm, 1995.
24. Cady et al Diagnosis and Treatment of Migraine, Mayo Clin Proc. 2002; 77:255-261.
25. Shand B. et. al. ‘Pilot Study on the Clinical Effects of Dietary Supplemention with Enzogenol®, A Flavonoid Extract of Pine Bark and Vitamin C’, Phytother. Res. 17, 490-494 (2003).
26. Burke B. E. et al, ‘Randomized, controlled trial of phytoestrogen in the treatment of menstrual migraine’ Biomedicine & Pharmacotherapy, 2002, 56(6) pp 283-288.
27. http://www.my.webmd.com/content/Article/26/1728_{—}58934.htm, ‘Herb holds promise for migraine prevention’ by Neil Osterweil
28. ‘Monograph Pelacites Hybridus (Butterbur)’ Altemative Medicine Review, Vol 6, No. 2, 2001, pp 207-209.
29. http://www.walgreens.com/store/product.jhtml?CATID=100077&id=prod393 841, ‘Migrahealth® Triple Therapy’
30. Nyholt D. R. et al. ‘Familial Typically Migraine—Linkage to Chromosome 19p13 and Evidence for Genetic Heterogeneity’, Neurology, May 1998 pp 1428-1432.
31. Silberstein S. D. ‘Practice Parameter: Evidence-Based Guidelines for Migraine Headache (An Evidence Based Review)’, Amer. Acad. Of Neurol. 2000.
32. http://www.drugstore.com/pharmacy/
33. ‘Food and Drink: Red Wine Headaches’ Harvard Health Letter, June 2002.
34. Gallagher R. M. ‘Migraine Medication Attributes Important for Patient Compliance: Concerns About Side Effects May Delay Treatment’, Headache: Journal of Head and Face Pain, Vol 43, Issue 1, Page 36-January 2003.
35. Braun L. ‘Feverfew’ Complementary Medicine, May/June 2004, pp 68-70.
36. Wood J. E. et al. ‘Antioxidant activity of procyanidin-containing plant extracts at different pHs’, Food Chemistry, 77 (2002) 155-161.
37. Rice-Evans, C. A. et al. (1996). “Structure-antioxidant activity relationships of flavonoids and phenolic acids.” Free Radic Biol Med 20(7): 933-56.
38. Galli, R. L. et al. (2002). “Fruit Polyphenolics and Brain Aging: Nutritional Interventions Targeting Age-related Neuronal and Behavioral Deficits.” Ann NY Acad Sci 959(1): 128-132.
39. Schroeter, H. et al. (2002). “MAPK signaling in neurodegeneration: influences of flavonoids and of nitric oxide.” Neurobiol of Aging 23(5): 861-880.
40. Ishige, K. et al. (2001). “Flavonoids protect neuronal cells from oxidative stress by three distinct mechanisms.” Free Radic Biol Med 30(4): 433-46.
41. Salah N., Miller N. J., et al. (1995). “Polyphenolic Flavanols as Scavengers of Aqueous Phase Radicals and as Chain-Breaking Antioxidants.” Archives of Biochemistry and Biophysics 322(2): 339-346.
42. Rice-Evans, C. A., N. J. Miller, et al. (1996). “Structure-antioxidant activity relationships of flavonoids and phenolic acids.” Free Radic Biol Med 20(7): 933-56.
43. Bagchi, D., C. K. Sen, et al. (2003). “Molecular mechanisms of cardioprotection by a novel grape seed proanthocyanidin extract.” Mutat Res 523-524: 87-97.
44. Sato, M., G. Maulik, et al. (1999). “Cardioprotective Effects of Grape Seed Proanthocyanidin Against lschemic Reperfusion Injury.” Journal of Molecular and Cellular Cardiology 31(6): 1289-1297.
45. Das, D. K., M. Sato, et al. (1999). “Cardioprotection of red wine: role of polyphenolic antioxidants.” Drugs Exp Clin Res 25(2-3): 115-20.
46. Nijveldt, R. J., E. van Nood, et al. (2001). “Flavonoids: a review of probable mechanisms of action and potential applications.” Am J Clin Nutr 74(4): 418-25.

1-19. (canceled)

20. A method of treatment of a human in need thereof by administration of a plant extract that includes a therapeutically effective dose of a mixture of flavonoids rich in proanthocyanidins to prevent migraine, reduce the frequency of migraine, or reduce the severity of migraine symptoms.

21. (canceled)

22. (canceled)

23. The method as claimed in claim 20 wherein the plant extract is administered in a form selected from the group including: a tablet; a capsule; a suppository; an injection; a suspension; a drink or tonic; a syrup; a powder; an ingredient in solid or liquid foods; a nasal spray; a sublingual wafer; a transdermal patch; a transdermal injection; and combinations thereof.

24. The method as claimed in claim 20 wherein the plant extract, also includes components selected from the group including: fillers; excipients; modifiers; humectants; stabilisers; emulsifiers; and other known formulation components.

25. The method as claimed in claim 20 wherein the plant extract also includes further antioxidant components.

26. The method as claimed in claim 25 wherein the further antioxidant components are vitamin C and vitamin E.

27. The method as claimed in claim 26 wherein the vitamin E used is a natural vitamin E.

28. The method as claimed in claim 20 wherein the flavonoid rich dose includes a mixture of compounds including: catechin; epicatechin; gallocatechin; dimers, trimers, oligomers and polymers of catechin and epicatechin; quercetin; dihydroquercetin; myricetin; astringenin; pinosylvin; taxifolin; stilbenes; hydroxylstilbenes; phenolic acids; and combinations thereof.

29. The method as claimed in claim 20 wherein the plant extract source is selected from: pine bark; grape seed; green tea; and combinations thereof.

30. The method as claimed in claim 20 wherein the plant extract source is bark from a plant.

31. The method as claimed in claim 20 wherein the plant extract source is bark of Pinus radiata.

32. The method as claimed in claim 29 wherein the pine bark extract dose exhibits antioxidant behaviour in vivo.

33. The method as claimed in claim 20 wherein the dose of flavonoids administered is varied depending on characteristics of the human to be treated selected from the group consisting of: metabolism, age, weight, gender, genetics, and combinations thereof.

34. The method as claimed in claim 20 wherein the plant extract is administered daily.

35. The method as claimed in claim 20 wherein the medicament is a capsule taken orally and the composition is administered in a dose regime of 1 to 10 capsules per day, each capsule containing approximately 120 mg pine bark extract, approximately 30 IU of natural vitamin E, and approximately 60 mg of vitamin C.

36. The method as claimed in claim 20 wherein the human has symptoms of regular, severe and acute migraine attacks.

37. The method as claimed in claim 20 wherein the symptoms are headaches.

38. The method as claimed in claim 20 wherein the symptoms are chronic daily headaches (CDH).

39. A composition containing:
a mixture of flavonoids rich in proanthocyanidins; and
Vitamin C, Vitamin E, or a combination thereof;
formulated to deliver a therapeutic dose for treatments selected from: preventing migraine symptoms; reducing the severity of migraine symptoms; or reducing the frequency of migraine symptoms.

40. (canceled)

41. (canceled)

42. A composition as claimed in claim 39 wherein the composition contains 120 to 1200 mg of flavanoid mixture rich in proanthocyanidins.

43. A composition as claimed in claim 39 wherein the composition contains 60 to 600 mg vitamin C.

44. A composition as claimed in claim 39 wherein the composition contains 30 to 300 IU of vitamin E.

45. The method as claimed in claim 29 wherein the plant extract is produced using a water-based process.
